SEQUENCE LISTING (34 sequences)

SEQ ID NO: 1 - DNA, 52 bp, Homo sapiens
  misc_feature (27)..(27): n is C or T
ctgaaccagggagctccccgaaggcgngaaccagggttgaattgcactccgc

SEQ ID NO: 2 - DNA, 52 bp, Homo sapiens
  misc_feature (27)..(27): n is C or G
gtccggggctccagcgagcggtagtgngagagcaggcagcgccggggggccg

SEQ ID NO: 3 - DNA, 52 bp, Homo sapiens
  misc_feature (27)..(27): n is absent or T
gcgagagcaggcagcgccggggggccntctgcgatcaccgtgcacaggaccc

SEQ ID NO: 4 - DNA, 52 bp, Homo sapiens
  misc_feature (27)..(27): n is absent or T
cgagagcaggcagcgccggggggcctnctgcgatcaccgtgcacaggaccca

SEQ ID NO: 5 - DNA, 52 bp, Homo sapiens; identical to SEQ ID NO: 4
  misc_feature (27)..(27): n is G or T

SEQ ID NO: 6 - DNA, 52 bp, Homo sapiens
  misc_feature (27)..(27): n is G or T
cttttgttttcctttctgtgagcaatntcacccaaattggaaccatgctgta

SEQ ID NO: 7 - DNA, 20 bp, Artificial Sequence
  synthetic sequence
tgtactgaaccagggagctc

SEQ ID NO: 8 - DNA, 17 bp, Artificial Sequence
  synthetic sequence
gatgcggccgagtgtct

SEQ ID NO: 9 - DNA, 14 bp, Artificial Sequence
  synthetic sequence
tggttcgcgccttc

SEQ ID NO: 10 - DNA, 15 bp, Artificial Sequence
  synthetic sequence
ctggttcacgccttc

SEQ ID NO: 11 - DNA, 16 bp, Artificial Sequence
  synthetic sequence
cctcagcgccttggca

SEQ ID NO: 12 - DNA, 17 bp, Artificial Sequence
  synthetic sequence
gatgcggccgagtgtct

SEQ ID NO: 13 - DNA, 16 bp, Artificial Sequence
  synthetic sequence
ctgctctcgcactacc

SEQ ID NO: 14 - DNA, 16 bp, Artificial Sequence
  synthetic sequence
ctgctctcccactacc

SEQ ID NO: 15 - DNA, 18 bp, Artificial Sequence
  synthetic sequence
ctccagcgagcggtagtg

SEQ ID NO: 16 - DNA, 19 bp, Artificial Sequence
  synthetic sequence
gcgcggagtgcaattcaac

SEQ ID NO: 17 - DNA, 13 bp, Artibeus aztecus
tcgcagaaggccc

SEQ ID NO: 18 - DNA, 13 bp, Artificial Sequence
  synthetic sequence
atcgcagcggccc

SEQ ID NO: 19 - DNA, 20 bp, Artificial Sequence
  synthetic sequence
tcagcctcgcctggtcctcc

SEQ ID NO: 20 - DNA, 20 bp, Artificial Sequence
  synthetic sequence
tgctgtgccttcacgctccg

SEQ ID NO: 21 - DNA, 28 bp, Artificial Sequence
  synthetic sequence
gggttaattttgtattgtttgttgtatt

SEQ ID NO: 22 - DNA, 25 bp, Artificial Sequence
  synthetic sequence
catatcccacctccaacttaaacta

SEQ ID NO: 23 - DNA, 26 bp, Artificial Sequence
  synthetic sequence
gttagtttaagttggaggtgggatat

SEQ ID NO: 24 - DNA, 23 bp, Artificial Sequence
  synthetic sequence
caacttctctacatcctcaaacc

SEQ ID NO: 25 - DNA, 24 bp, Artificial Sequence
  synthetic sequence
ggattttaggatatttagaggatt

SEQ ID NO: 26 - DNA, 17 bp, Artificial Sequence
  synthetic sequence
atcctaaaactactaac

SEQ ID NO: 27 - DNA, 23 bp, Artificial Sequence
  synthetic sequence
ttgtatagtttgggatttttgga

SEQ ID NO: 28 - DNA, 21 bp, Artificial Sequence
  synthetic sequence
caaaatcaatcacaaaaaaaa

SEQ ID NO: 29 - DNA, 25 bp, Artificial Sequence
  synthetic sequence
tttttttgtgattgattttgagttt

SEQ ID NO: 30 - DNA, 23 bp, Artificial Sequence
  synthetic sequence
ctataccccctaaaaccctaacc

SEQ ID NO: 31 - DNA, 22 bp, Artificial Sequence
  synthetic sequence
gttttagggggtataggggttg

SEQ ID NO: 32 - DNA, 25 bp, Artibeus aztecus
aaaccctctctttaacttccctaac

SEQ ID NO: 33 - DNA, 26 bp, Artificial Sequence
  synthetic sequence
ttagggaagttaaagagagggtttag

SEQ ID NO: 34 - DNA, 26 bp, Artificial Sequence
  synthetic sequence
actcccactctctctcccactatata

(19) United States
(12) Patent Application Publication
(10) Pub. No.: US 2014/0154209 A1
(43) Pub. Date: Jun. 5, 2014

(54) POLYMORPHISMS ASSOCIATED WITH NON-RESPONSE TO A HEPATITIS C TREATMENT OR SUSCEPTIBILITY TO NON-SPONTANEOUS HEPATITIS C CLEARANCE

(71) Applicants: Stephanie Bibert, Divonne-Les-Bains (FR); Pierre-Yves Bochud, Grandvaux (CH)

(72) Inventors: Stephanie Bibert, Divonne-Les-Bains (FR); Pierre-Yves Bochud, Grandvaux (CH)

(73) Assignee: Centre Hospitalier Universitaire Vaudois, Lausanne (CH)

(21) Appl. No.: 14/125,885

(22) PCT Filed: Jun. 29, 2012
(86) PCT No.: PCT/IB2012/053316

§ 371 Date: Dec. 12, 2013

Related U.S. Application Data
(60) Provisional application No. 61/457,895, filed on Jun. 30, 2011.

(30) Foreign Application Priority Data
Nov. 17, 2011 (IB) PCT/IB2011/055157

(51) Int. Cl. C12Q 1/70

(52) U.S. Cl. CPC C12Q 1/707
USPC 424/85.5; 435/5; 424/85.7; 514/43

(57) ABSTRACT

The present invention relates to in vitro methods of determining a susceptibility to non-response to a hepatitis C treatment or a susceptibility to non spontaneous hepatitis C clearance in a subject infected with hepatitis C.

### FIELD OF THE INVENTION

The present invention relates to in vitro methods of determining a susceptibility to non-response to a hepatitis C treatment or a susceptibility to non spontaneous hepatitis C clearance in a subject infected with hepatitis C.

### BACKGROUND OF THE INVENTION

The hepatitis C virus (HCV) is a positive-stranded RNA virus that belongs to the family of Flaviviridae. Six distinct genotypes have been described, with genotypes 1 being the most frequent in the United States and in Western Europe. Spontaneous clearance of the virus is observed in ˜30% of infected individuals, while the others develop chronic infection. Chronic hepatitis C affects ˜3% of the world population. Morbidity in chronic infection mainly results from the development of liver fibrosis and cirrhosis, with complications such as hepatocellular carcinoma and liver failure. The standard treatment of chronic hepatitis C is a combination of pegylated interferon-α and ribavirin (PEG-IFN-α/RBV) which results in sustained virological response (SVR) in ˜50% of patients infected with genotype 1 or 4, and 70-90% of those infected genotype 2 and 3 patients.

Recent genome-wide association studies (GWAS) showed that single nucleotide polymorphisms (SNPs) nearby the IL28B gene are strongly associated with spontaneous {Rauch et al., 2010} and treatment-induced {Ge et al., 2009; Suppiah et al., 2009; Tanaka et al., 2009; Rauch et al., 2010} clearance of HCV infection. IL28B encodes interferon-λ3 (IFN-λ3), a cytokine that was shown to have antiviral properties both in vitro and in vivo {Ank et al., 2006; Robek et al., 2005}. IFN-λ3 belongs to the family of type III interferons, which are structurally close to the IL-10 family and functionally related to type I interferons. After the publication of these GWAs, numerous investigators confirmed and extended our knowledge on the importance of IL28B SNPs in the natural course and treatment of HCV infection (reviewed in Lange et al., 2011). IL28B genetic tests are now commercially available to help treatment decision (http://www.questdiagnostics.com/). Two major SNPs were identified from different studies, namely rs12979860 and rs8099917 {Suppiah et al., 2009; Tanaka et al., 2009; Rauch et al., 2010; Ge et al., 2009; Thomas et al., 2009}, which have some level of linkage disequilibrium (R2˜0.50 in Caucasians), but different allele frequencies (0.35-0.38 for rs12979860 and 0.21-0.26 for rs8099917 in Caucasian HCV-infected patients).

The mechanisms by which IL28B polymorphisms influence HCV clearance are unclear. It is unknown whether rs12979860 and rs8099917 exert direct biological effects or are in linkage disequilibrium with other functional polymorphisms. Some investigators reported that whole blood or peripheral blood mononuclear cells (PBMCs) from individuals carrying the SNPs had a lower expression of IL28 than those carrying the WT alleles {Suppiah et al., 2009; Tanaka et al., 2009}, but these observations were controversal when using biopsies from HCV-infected livers {Dill et al., 2011; Honda et al., 2010; Urban et al., 2010}. Other investigators performed gene mapping but failed to detect new SNPs with a stronger genetic effect {di Iulio et al., 2011} or with a clear functional mechanism.

Despite the above-mentioned approach, there is still a profound need to develop an effective predictive method of determining a susceptibility to non-response to an anti hepatitis C therapy in a subject suffering from chronic hepatitis C, or a susceptibility to spontaneous or non spontaneous hepatitis C clearance in a subject acutely infected with hepatitis C virus.

### SUMMARY OF THE INVENTION

This object has been achieved by providing a method of determining a susceptibility of non response to a hepatitis C treatment in a subject suffering from chronic hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

A further object of the present invention is to provide a method of determining a susceptibility to non-spontaneous hepatitis C clearance in a subject infected with hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

The invention also provides a kit for determining a susceptibility to non-response to a hepatitis C treatment in a subject suffering from chronic hepatitis C according to the method of any of claims 1 to 15, said kit comprising i) reagents for selectively detecting the presence or absence of at least one methylation associated polymorphism within the 5′ region upstream front the transcription start of the IL28B/A locus ii) instructions for use.

The invention further provides a kit for determining a susceptibility to non-spontaneous hepatitis C clearance in a subject infected with hepatitis C according to the method of any of claims 16 to 23, said kit comprising i) reagents for selectively detecting the presence or absence of at least one methylation associated polymorphism within the 5′ region upstream from the transcription start of the IL28B/A locus and ii) instructions for use.

The invention also provides a method of treating a patient for hepatitis C, comprising i) determining the presence or absence of at least one methylation associated polymorphism within the 5′ region upstream from the transcription start of the IL28B/A locus in a nucleic acid sample isolated from a biological sample obtained from said patient, ii) and treating the patient based upon whether said one methylation associated polymorphism within the 5′ region upstream from the transcription start of the IL28B/A locus is associated with increased susceptibility to non-response to hepatitis C treatment.

Another object of the invention is to provide a method of treating a patient for hepatitis C, comprising i) determining the presence or absence of at least one methylation associated polymorphism within the 5′ region upstream from the transcription start of the IL28B/A locus in a nucleic acid sample isolated from a biological sample obtained from said patient, ii) determining the HCV viral genotype in a nucleic acid sample isolated from a biological sample obtained from said patient, iii) and treating the patient based upon whether said one methylation associated polymorphism within the 5′ region upstream from the transcription start of the IL28B/A locus is associated with increased susceptibility to non-response to hepatitis C treatment.

Still a further object of the present invention is a method of assessing a susceptibility to non-response to a hepatitis C treatment in a subject suffering from hepatitis C, said method comprising: i) distinguishing in said subjects those having a susceptibility to non-response to a hepatitis C treatment by determining the presence or absence of at least one methylation associated polymorphism within the 5′ region upstream from the transcription start of the IL28B/A locus in a nucleic acid sample isolated from a biological sample obtained from said patient, the presence of said at least one methylation associated polymorphism being an indication that said subject has an increased susceptibility to non-response to a hepatitis C treatment, ii) establishing a hepatitis C treatment regimen.

This invention also provides a method of assessing a susceptibility to non-response to a hepatitis C treatment in a subject suffering from hepatitis C, said method comprising: i) distinguishing in said subjects those having a susceptibility to non-response to a hepatitis C treatment by determining
the presence or absence of at least one methylation associated polymorphism within the 5′ region upstream from the transcription start of the IL28B/A locus in a nucleic acid sample isolated from a biological sample obtained from said subject, the presence of said one methylation associated polymorphism being an indication that said subject has an increased susceptibility to non-response to a hepatitis C treatment, and
the HCV viral genotype, the presence of genotype 1 and/or 4 being an indication that said subject has an increased susceptibility to non-response to a hepatitis C treatment,
   ii) establishing a hepatitis C treatment regimen.

### DESCRIPTION OF THE FIGURES

FIG. 1. A. Schematic representation of the region upstream IL28B start codon showing previously identified SNPs (rs8099917 and rs12979860), a ˜1000 bp CpG region delimited using the cpgplot software and methlylation pattern-associated polymorphisms (rs4803221 and chr19: 39739154-5 TT/−G). B. Amplicons (Al-7) used for bisulfite sequencing and for unmethylated reference sequence (Aun).

FIG. 2. Chromatograms for unmethylated reference and bisulfite treated DNA sequences. The rs4803221 C to G substitution (upper left) induced a loss of methylation site (lower left, indicated by an arrow). The chr19:39739154-5 TT to −G deletion/substitution (upper right) induced a gain of methylation site (lower right, indicated by an arrow). As opposed to methylated cytosine residues (indicated by stars), unmethylated cytosine residues (C) are replaced by thymine (T) following bisulfite treatment and subsequent PCR amplification.

### DETAILED DESCRIPTION OF THE INVENTION

A number of studies showed that polymorphisms nearby IL28B are strong predictors of spontaneous and treatment-induced clearance of hepatitis C infection {Lange et al., 2011}. However, the causal variant and functional mechanisms involved in this association are still unknown. Investigators failed to detect a coding non-synonymous SNP affecting the protein expression and/or its function. Studies showed that polymorphisms in close proximity with IL28B are associated by differential expression of the gene, suggesting that the causal variants may be located outside the coding region.

Although the methylation profile was overall conserved among patients, the Applicants have shown that methylation associated polymorphisms located within the 5′ region upstream from the transcription start of the IL28B/A locus are better predictor of spontaneous and treatment-induced clearance of hepatitis C infection than polymorphisms previously described.

Accordingly, the invention provides a method of determining a susceptibility of non response to a hepatitis C treatment in a subject suffering from chronic hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

The invention also provides a method of determining a susceptibility to non-spontaneous hepatitis C clearance in a subject infected with hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Alternatively, the term “comprise” or “comprising” also embraces the term “consist” or “consisting”, respectively.

As used in the specification and claims, the singular form “a”, “an” and “the” include plural references unless the context clearly dictates otherwise.

As used herein “Sustained viral response” was defined as an undetectable viremia more than 24 weeks after treatment was terminated.

As used herein, “at least one” means “one or more”, “two or more”, “three or more”, etc.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of a hepatitis C treatment. However, in other embodiments, the subject can be a normal subject. The terms “subject” or “patient” do not denote a particular age or sex. Thus, adult, infant and newborn subjects, whether male or female, are intended to be covered. Alternatively, said subject suffering from hepatic C, or patient suffering from hepatic C, is co-infected with the human immunodeficiency virus (HIV), preferably HIV-1 or HIV-2.

As used herein the term “susceptibility” refers to the likelihood, for the subject, or a predisposition not to respond to hepatitis C treatment or to a predisposition, for the subject, to a non-spontaneous hepatitis C clearance.

An “allele”, as used herein, refers to one specific form of a genetic sequence or a single nucleotide position within a genetic sequence (such as a gene) within a cell, an individual or within a population, the specific form differing from other forms of the same gene in the sequence of at least one, and frequently more than one, variant sites within the sequence of the gene. The sequence may or may not be within a gene. The sequences at these variant sites that differ between different alleles are termed “variances”, “polymorphisms”, or “mutations”. At each autosomal specific chromosomal location or “locus”, an individual possesses two alleles, one inherited from one parent and one from the other parent, for example one from the mother and one from the father.

“Polymorphism”, as used herein, refers to the occurrence of two or more genetically determined alternative sequences or alleles in a population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, copy number variations (CNV) and insertion elements such as Alu. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. A particular kind of polymorphism, called a single nucleotide polymorphism, or SNP, is a small genetic change or variation that can occur within a person's DNA sequence. The genetic code is specified by the four nucleotide “letters” A (adenine), C (cytosine), T (thymine), and G (guanine) SNP variation occurs when a single nucleotide, such as an A, replaces one of the other three nucleotide letters—C, G, or T.

As used herein, a “methylation associated polymorphism” refers to a polymorphism that is associated with a change of methylation, e.g. a loss or a gain of methylation.

The primary target of methylation is the two-nucleotide sequence Cytosine-Guanine (a ‘CpG site’); within this context cytosine (C) can undergo a simple chemical modification to become 5-methyl-cytosine. In the human genome, the CG sequence is much rarer than expected except in certain relatively dense clusters called “CpG islands”. CpG islands, also called CpG regions, are frequently associated with gene promoters, and it has been estimated that more than half of the human genes have CpG islands). In the present invention, the at least one methylation associated polymorphism is preferably in a CpG region.

In case the subject suffering from chronic hepatitis C is a human, then preferably said CpG region is located on chromosome 19 between base pairs, (as determined by emboss using Genome Build 37.1), 39738000 and 39739500, most preferably between base pairs 39738200 and 39739300, even more preferably between base pairs 39738515 and 39739270.

The term “hepatitis C virus” or “HCV” is used herein to define an RNA viral species of which pathogenic strains cause hepatitis C, also known as non-A, non-B hepatitis. Based on genetic differences between HCV isolates, the hepatitis C virus species is classified into six genotypes (1, 2, 3, 4, 5 or 6) with several subtypes within each genotype. Subtypes are further broken down into quasi species based on their genetic diversity. The preponderance and distribution of HCV genotypes varies globally. Genotypes 1 and 4 are generally less responsive to interferon-based treatment than are the other genotypes (2, 3, 5 and 6). It is to be noted that genotypes 5 and 6 are rare in the population.

“Hepatitis C” is an infectious disease affecting the liver, caused by the hepatitis C virus (HCV). The infection is often asymptomatic, but once established, chronic hepatitis C infection can progress to scarring of the liver (fibrosis), and advanced scarring (cirrhosis) which is generally apparent after many years. In some cases, those with cirrhosis will go on to develop liver failure or other complications of cirrhosis, including liver cancer (hepatocellular carcinoma).

“Chronic hepatitis C” is defined as infection with the hepatitis C virus persisting for more than six months. Clinically, it is often asymptomatic (without symptoms) and it is mostly discovered accidentally. The natural course of chronic hepatitis C varies considerably from one person to another. Although almost all people infected with HCV have evidence of inflammation on liver biopsy, the rate of progression of liver scarring (fibrosis) shows significant variability among individuals. Accurate estimates of the risk over time are difficult to establish because of the limited time that tests for this virus have been available.

Soon after the hepatitis C virus (HCV) was identified, a number of cross-sectional studies in people with antibodies to the virus demonstrated that some appeared to show “spontaneous hepatitis C clearance”, while others maintained a state of viraemia. Since then, a number of investigators have endeavoured to characterize the pathogenesis of hepatitis C infection, including the rate, time course and predictors of spontaneous viral clearance. Estimates of clearance rates have ranged from 10 to 50%, and the duration of time to clearance has been found to be as long as 3 years in some cases. Authoritative clinical reviews have generally quoted clearance rates as low as 10-15%.

“Non-spontaneous hepatitis C clearance” refers herein to a situation where a subject would not present spontaneous clearance, such that the infection would evolve into chronic hepatitis C. For the sake of clarity, it does not refer to a treatment-induced clearance.

“IL28B/A locus” generally refers, in humans, to a genomic DNA region located within a 80 kb region in the long arm of chromosome 19 encoding two cytokine genes, i.e. IL28B and IL28A (which belong to the IFN, family). These genes have 6 exons for IL-28A (IFN2λ2) and IL-28B (IFNλ3). They encode 20 kDa secreted monomeric proteins. It has recently been reported that IL28B, IL28A cytokines could be an interesting substitute to IFNα for the treatment of HCV-infected patients who are or become resistant to IFNα.

In a preferred embodiment, the at least one methylation associated polymorphism, which presence is an indication that said subject has an increased susceptibility to non response to a hepatitis C treatment and/or to non spontaneous clearance, is selected among the non limiting examples of SNP polymorphisms of Table 1, below:

TABLE 1

| | Geno- | | Spontaneous | Response |
|---|---|---|---|---|
| SNP | type | Methylation | Clearance | to treatment. |
| rs12979860 | C/C | NA | reference | reference |
| | C/T | NA | less likely | less likely |
| | T/T | NA | less likely | less likely |
| rs4803221 | C/C | Yes/Yes | reference | reference |
| | C/G | Yes/No | less likely | less likely |
| | G/G | No/No | less likely | less likely |
| chr19: 39739154-5 | TT/TT | No/No | reference | reference |
| TT/-G | TT/-G | No/Yes | less likely | less likely |
| | -G/-G | Yes/Yes | less likely | less likely |
| rs8099917 | TT | NA | reference | reference |
| | TG | NA | less likely | less likely |
| | GG | NA | less likely | less likely |

Most preferably, the at least one methylation associated polymorphism is selected from the group comprising rs67272382 T/−, rs67272382−/−, rs74597329 T/G, rs74597329 G/G, rs4803221 C/G and rs4803221 G/G. The dbSNP database indicates that rs67272383 has been merged into rs67272382 (http://www.ncbi.nlm.nih.gov/snp?term=rs67272383)
Although both polymorphisms rs67272382 (located at position 39739154 on chromosome 19) and rs74597329 (located at position 39739155 on chromosome 19) were attributed specific SNP numbers, they were always present together in sequenced patients of the present invention and thus were in complete or strong linkage disequilibrium one each other. Accordingly, for the ease of the reader, these polymorphisms were called chr19: 39739154-5 TT/−G deletion substitution in the present description.
The nucleic acid sequences of the above-mentioned methylation associated polymorphisms are as follows:

TABLE 2

| rs number | Strand | | Minor | MAF CEU |
|---|---|---|---|---|
| (SEQ ID No) | +/− | Sequence | allele | (hapmap) |
| rs12979860 | + | CTGAACCAGGGAGCTCCCCGAAGGCG[C/T]GAACCAGGGTTGAATTGCACTCCGC | T | 0.331 |
| SEQ ID No 1 | | | | |
| rs4803221 | + | GTCCGGGGCTCCAGCGAGCGGTAGTG[C/G]GAGAGCAGGCAGCGCCGGGGGGCCG | G | 0.119 |
| SEQ ID No 2 | | | | |
| rs67272382 | + | GCGAGAGCAGGCAGCGCCGGGGGGCC[—/T]TCTGCGATCACCGTGCACAGGACCC | — | |
| SEQ ID No 3 | | | | |
| rs11322783 | + | CGAGAGCAGGCAGCGCCGGGGGGCCT[—/T]CTGCGATCACCGTGCACAGGACCCA | — | |
| SEQ ID No 4 | | | | |
| rs74597329 | + | CGAGAGCAGGCAGCGCCGGGGGGCCT[G/T]CTGCGATCACCGTGCACAGGACCCA | G | |
| SEQ ID No 5 | | | | |
| rs8099917 | + | CTTTTGTTTTCCTTTCTGTGAGCAAT[G/T]TCACCCAAATTGGAACCATGCTGTA | G | 0.101 |
| SEQ ID No 6 | | | | |

The methylation associated polymorphism rs4803221 was a C to G (C/G) resulting in a loss of methylation. The second was a TT to −G deletion/substitution resulting in a gain of methylation (chr19: 39739154-5 TT/−G). Both were located ˜500 bp upstream rs12979860. Genotyping in the whole cohort showed that chr19: 39739154-5 TT/−G polymorphism was a better predictor of spontaneous and treatment-induced clearance of hepatitis C infection than rs12979860 and rs8099917. Chr19: 39739154-5 TT/−G allowed for better treatment response rate discrimination in patients infected with genotype 1 and/or 4.

Chr19: 39739154-5 TT/−G was the only IL28B polymorphism that tended to be associated with response to treatment in patients infected with HCV genotypes 2 and 3, although this association was present only when using the recessive model, and disappeared in the multivariate model. As previously reported, the small prediction is probably due to the high overall response rate and small number of patients infected with genotypes 2 and 3.
Usually, the at least one methylation associated polymorphism described supra is in complete or strong linkage disequilibrium with at least one SNP selected from the group comprising rs12979860 and rs8099917.
For example, the chr19:39739154-5 TT/−G methylation associated polymorphism is in stronger linkage disequilibrium with rs12979860 (R2=0.74) than with rs8099917 (R2=0.33). Both rs12979860 and chr19:39739154-5 TT/−G, but not rs8099917, are located in a ˜1000 bp CpG island upstream IL28B start codon.

“Linkage disequilibrium” (LD) describes a situation in which some combinations of alleles or genetic markers occur more or less frequently in a population than would be expected from a random formation of haplotypes from alleles based on their frequencies. When a particular allele at one locus is found together on the same chromosome with a specific allele at a second locus (more often than expected if the loci were segregating independently in a population) the loci are in disequilibrium. This concept of LD is formalized by one of the earliest measures of disequilibrium to be proposed (symbolized by D). D, in common with most other measures of LD, quantifies disequilibrium as the difference between the observed frequency of a two-locus haplotype and the frequency it would be expected to show if the alleles are segregating at random. Adopting the standard notation for two adjacent loci-A and B, with two alleles (A, a and B, b) at each locus-the observed frequency of the haplotype that consists of alleles A and B is represented by PAB. Assuming the independent assortment of alleles at the two loci, the expected halotype frequency is calculated as the product of the allele frequency of each of the two alleles, or PA×PB, where PA is the frequency of allele A at the first locus and PB is the frequency of allele B at the second locus. So, one of the simplest measures of disequilibrium is D=PAB-PA×PB. LD is created when a new mutation occurs on a chromosome that carries a particular allele at a nearby locus, and is gradually eroded by recombination. Recurrent mutations can also lessen the association between alleles at adjacent loci. The importance of recombination in shaping patterns of LD is acknowledged by the moniker of “linkage”. The extent of LD in populations is expected to decrease with both time (t) and recombinational distance (r, or the recombination fraction) between markers. Theoretically, LD decays with time and distance according to the following formula, where D0 is the extent of disequilibrium at some starting point and Dt, is the extent of disequilibrium t generation later: Dt=(1−r)tD0

A wide variety of statistics have been proposed to measure the amount of LD, and these have different strengths, depending on the context. Although the measure D has the intuitive concepts of LD, its numerical value is of little use for measuring the strength of and comparing levels of LD. This is due to the dependence of D on allele frequencies. The two most common measures are the absolute value of D′ and R2. Strong LD was defined as a R2>0.7.

The absolute value of D′ is determined by dividing D by its maximum possible value, given the allele frequencies at the two loci. The case of D′=1 is known as complete LD. Values of D′<1 indicate that the complete ancestral LD has been disrupted. The magnitude of values of D′<1 has no clear interpretation. Estimates of D′ are strongly inflated in small samples. Therefore, statistically significant values of D′ that are near one provide a useful indication of minimal historical recombination, but intermediate values should not be used for comparisons of the strength of LD between studies, or to measure the extent of LD.

The measure R2 is in some ways complementary to D′. R2 is equal to D2 divided by the product of the allele frequencies at the two loci. Hill and Roberson deduced that E [R2]=1/1+4Nc where c is the recombination rate in morgans between the two markers and N is the effective population size. This equation illustrates two important properties of LD. First, expected levels of LD are a function of recombination. The more recombination between two sites, the more they are shuffled with respect to one another, decreasing LD. Second, LD is a function of N, emphasizing that LD is a property of populations.

It has also been found that the association between the presence of SNPs and the susceptibility to non response to hepatitis C treatment or to non spontaneous clearance was observed both in HCV mono-infected and in HCV/HIV co-infected individuals.

A number of methods are available for analyzing the presence or absence of at least one methylation associated polymorphism within the 5′ region upstream from the transcription start of the IL28B/A locus, in a nucleic acid sample isolated from a biological sample obtained from a subject. Assays for detection of polymorphisms or mutations fall into several categories, including but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention, and are described in relationship to detection of the various methylation associated polymorphism found in the 5′ region.

In one aspect of the present invention, the methylation associated polymorphisms like SNPs that are detected using a direct sequencing technique. In these assays, DNA samples are first isolated from a subject using any suitable method. In some embodiments, the region of interest is cloned into a suitable vector and amplified by growth in a host cell (e.g., a bacterium). In other embodiments, DNA in the region of interest is amplified using the Polymerase Chain Reaction (PCR).
Following amplification, DNA in the region of interest (e.g., the region containing the SNP) is sequenced using any suitable method, including but not limited to manual sequencing using radioactive marker nucleotides, or automated sequencing. The results of the sequencing are displayed using any suitable method. The sequence is examined and the presence or absence of a given SNP is determined.
In one aspect of the present invention, the methylation associated polymorphisms, like SNPs, are detected using a PCR-based assay. In some embodiments, the PCR assay comprises the use of oligonucleotide primers (“primers”) to amplify a fragment containing the repeat polymorphism of interest. Amplification of a target polynucleotide sequence may be carried out by any method known to the skilled artisan. See, for instance. Amplification methods include, but are not limited to, PCR including real time PCR (RT-PCR), strand displacement amplification, strand displacement amplification using Phi29 DNA polymerase (U.S. Pat. No. 5,001,050), transcription-based amplification, self-sustained sequence replication (“3SR”); the Q.beta. replicase system, nucleic acid sequence-based amplification (“NASBA”), the repair chain reaction (“RCR”), and boomerang DNA amplification (or “BDA”). PCR is the preferred method of amplifying the target polynucleotide sequence.
PCR may be carried out in accordance with techniques known by the skilled artisan. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained.
The amplified target polynucleotide may be used in one of the detection assays described elsewhere herein to identify the GT-repeat polymorphism present in the amplified target polynucleotide sequence.
In one aspect of the present invention, the methylation associated polymorphisms like SNPs are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.
In one aspect of the present invention, fragment sizing analysis is carried out using the Beckman Coulter CEQ 8000 genetic analysis system, a method well-known in the art for microsatellite polymorphism determination.
In one aspect of the present invention, the methylation associated polymorphisms like SNPs are detected using a restriction fragment length polymorphism assay (RPLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining and compared to controls (wild-type).
In one aspect, polymorphisms are detected using a CLEAVASE fragment length polymorphism assay (CFLP; Third Wave Technologies, Madison, Wis.; see e.g., U.S. Pat. No. 5,888,780). This assay is based on the observation that, when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.
The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given polymorphism. The CLEAVASE enzyme treated PCR products are separated, detected (e.g., by agarose gel electrophoresis), visualized (e.g., by ethidium bromide staining) and compared to controls (wild-type).
In other aspects of the present invention, the methylation associated polymorphisms like SNPs are detected by hybridization assay. In a hybridization assay, the presence or absence of a given polymorphism or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.
In a preferred aspect, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, polymerase chain reaction (PCR) with labeled primers or labeled nucleotides will provide a labeled amplification product. In another embodiment, transcription amplification using a labeled nucleotide (e.g. fluorescein-labeled UTP and/or CTP) incorporates a label into the transcribed nucleic acids.
Alternatively, a label may be added directly to the original nucleic acid sample (e.g., mRNA, polyA mRNA, cDNA, genomic DNA etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase (TdT).
Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies; magnetic beads (e.g., Dynabeads™); fluorescent dyes (e.g., fluorescein, Texas Red, rhodamine, green fluorescent protein, and the like); radiolabels (e.g., ³H, ¹²⁵l, ³⁵S, ¹⁴C, or ³²P); phosphorescent labels; enzymes (e.g., horse radish peroxidase, alkaline phosphatase and others commonly used in an ELISA); and calorimetric labels such as colloidal gold or colored glass or plastic (e.g., polystyrene, polypropylene, latex, etc.) beads.
Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.
The label may be added to the target nucleic acid(s) prior to, or after the hybridization. So-called “direct labels” are detectable labels that are directly attached to or incorporated into the target nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids. See Tijssen, 1993, Laboratory Techniques in Biochemistry and Molecular Biology, Vol. 24: Hybridization with Nucleic Acid Probes, which is hereby incorporated by reference in its entirety for all purposes.
In one aspect, hybridization of a probe to the sequence of interest (e.g., methylation associated polymorphisms like SNP) is detected directly by visualizing a bound probe (e.g., a Northern or Southern assay; See e.g., Ausabel et al. (Eds.), 1991, Current Protocols in Molecular Biology, John Wiley & Sons, NY). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.
In one aspect of the present invention, the methylation associated polymorphisms like SNPs are detected using a DNA chip hybridization assay. In this assay, a series of oligonucleotide probes are affixed to a solid support. The oligonucleotide probes are designed to be unique to a given single nucleotide polymorphism. The DNA sample of interest is contacted with the DNA “chip” and hybridization is detected.
In some embodiments, the DNA chip assay is a GeneChip (Affymetrix, Santa Clara, Calif.; see e.g., U.S. Pat. No. 6,045,996) assay. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.
The nucleic acid to be analyzed is isolated from a biological sample obtained from the subject, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.
In another aspect, a DNA microchip containing electronically captured probes (Nanogen, San Diego, Calif.) is utilized (see e.g., U.S. Pat. No. 6,068,818). Through the use of microelectronics, Nanogen's technology enables the active movement and concentration of charged molecules to and from designated test sites on its semiconductor microchip. DNA capture probes unique to a given polymorphism or mutation are electronically placed at, or “addressed” to, specific sites on the microchip. Since DNA has a strong negative charge, it can be electronically moved to an area of positive charge.
First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.
A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.
In still another aspect, an array technology based upon the segregation of fluids on a flat surface (chip) by differences in surface tension (ProtoGene, Palo Alto, Calif.) is utilized (see e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on an X/Y translation stage under a set of four piezoelectric nozzles, one for each of the four standard DNA bases. The translation stage moves along each of the rows of the array, and the appropriate reagent is delivered to each of the reaction site. For example, the A amidite is delivered only to the sites where amidite A is to be coupled during that synthesis step and so on. Common reagents and washes are delivered by flooding the entire surface followed by removal by spinning.
DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).
In yet other aspects, a “bead array” is used for the detection of SNPs (Illumina, San Diego, Calif.; see e.g., PCT Publications WO99/67641 and WO00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given polymorphism or mutation. Batches of beads are combined to form a pool specific to the array. To perform an assay, the bead array is contacted with a prepared subject sample (e.g., DNA). Hybridization is detected using any suitable method like Enzymatic Detection of Hybridization
In some aspects of the present invention, genomic profiles are generated using an assay that detects hybridization by enzymatic cleavage of specific structures (INVADER assay, Third Wave Technologies; see e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the dequenched fluorescein labeled product may be detected using a standard fluorescence plate reader.
The INVADER assay detects specific mutations and polymorphisms in unamplified genomic DNA. The isolated DNA sample is contacted with the first probe specific either for a polymorphism/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.
In some aspects, hybridization of a bound probe is detected using a TaqMan assay (PE Biosystems, Foster City, Calif.; see e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.
In some aspects, a MassARRAY system (Sequenom, San Diego, Calif.) is used to detect polymorphisms (see e.g., U.S. Pat. No. 6,043,031). DNA is isolated from blood samples using standard procedures. Next, specific DNA regions containing the polymorphism of interest are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.
Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

Usually, the “nucleic acid sample” of the invention is isolated from a biological sample obtained from the subject, such as whole blood, serum, semen, saliva, tears, urine, fecal material, sweat, buccal smears, skin, and biopsies of muscle, liver, brain tissue, nerve tissue and hair. The nucleic acid sample may be a portion of a gene, a regulatory sequence, genomic DNA, cDNA, and RNA (including mRNA, mi RNA and rRNA).

Genomic DNA samples are usually amplified before being brought into contact with a probe. Genomic DNA can be obtained from any biological sample. Amplification of genomic DNA containing a SNP generates a single species of nucleic acid if the individual from whom the sample was obtained is homozygous at the polymorphic site, or two species of nucleic acid if the individual is heterozygous.
RNA samples also are often subject to amplification. In this case, amplification is typically preceded by reverse transcription. Amplification of all expressed mRNA can be performed as described in, for example, in [39] and [40] which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.
The genotype of an individual polymorphism comprises the sum of at least two alleles and may be homozygous (i.e. comprising identical alleles) or heterozygous (i.e. comprising different alleles).

In some aspect of the invention, the isolated nucleic acid sample of the present invention can be produced or synthesized using conventional nucleic acid synthesis or by recombinant nucleic acid methods known in the art (2001, Molecular Cloning: A Laboratory Manual, Cold Spring Harbor Laboratory Press, New York) and Ausubel et al. (2001, Current Protocols in Molecular Biology, Green & Wiley, New York).

The Applicants of the present invention have also shown the association of patient's methylation associated polymorphism and certain type of HCV viral genotype are associated either with increased susceptibility to non-response to hepatitis C treatment and/or with increased susceptibility to non-spontaneous hepatitis C clearance. For example, Chr19: 39739154-5 TT/−G allowed for better treatment response rate discrimination in patients infected with genotype 1 and/or 4. On the other hand, Chr19: 39739154-5 TT/−G was the only IL28B polymorphism that tended to be associated with response to treatment in patients infected with HCV genotypes 2 and 3, although this association was present only when using the recessive model, and disappeared in the multivariate model.

Accordingly, the method of the invention further comprises determining the HCV viral genotype in a nucleic acid sample isolated from a biological sample of said subject.

In one aspect, the nucleic acid sample useful for the determination of the viral genotype and the nucleic acid sample useful for the determination of the methylation associated polymorphism, as described herein, are isolated from the same biological sample obtained from the subject. The biological sample is then prepared on one hand for the isolation of the nucleic acid sample useful for determining the presence or absence of the at least one methylation associated polymorphism of the invention and on the other hand for determining the HCV viral genotype.

In another aspect, the nucleic acid sample useful for the determination of the viral genotype and the nucleic acid sample useful for the determination of the at least one methylation associated polymorphism, as described herein, are isolated from two different biological samples obtained from the subject. In this case, the first biological sample is then prepared for the isolation of the nucleic acid sample useful for determining the presence or absence of the at least one methylation associated polymorphism of the invention whereas the second biological sample is prepared for the isolation of the nucleic acid sample useful for determining the HCV viral genotype. These two biological samples can be of same nature (e.g. whole blood in the two cases) or different (e.g. whole blood and liver biopsy).

The HCV nucleic acid, usually RNA, to be analyzed is generally isolated, reverse transcribed into cDNA and amplified, for example, by PCR as described in WO 96/14839 and WO 97/01603. Any other techniques known in the art can be applied.

Generally, the hepatitis C treatment of the invention is either an interferon based treatment or a non interferon based treatment.

In one embodiment, the interferon based treatment is selected from the group comprising IFNα, IFNλ or any pegylated-interferon. Usually, said interferon based treatment is combined with ribavirin. Alternative combinations may include ribavirin and/or antiprotease drugs and/or other antiviral drugs and/or any combination thereof.

Alternatively also, ribavirin is absent and the interferon based treatment is combined with an antiprotease drug and/or an antiviral drug and/or any combination thereof.

In another embodiment, the non-interferon based treatment is selected from the group comprising an antiprotease drug, an antiviral drug and/or any combination thereof.

An example of an antiviral drug of the invention is a cyclophylin inhibitor such as alisporivir.

Examples of an antiprotease drug are selected from the group comprising

1) NS3-4A protease inhibitors such as boceprevir,

2) HCV NS5B inhibitors, such as nucleoside or non-nucleoside NS5B inhibitors, and

3) HCV NS5A inhibitors.

Further encompassed in the present invention is a method of assessing a susceptibility to non-response to a hepatitis C treatment in a subject suffering from hepatitis C, said method comprising: i) distinguishing in said subjects those having a susceptibility to non-response to a hepatitis C treatment by determining the presence or absence of at least one methylation associated polymorphism within the 5′ region upstream from the transcription start of the IL28B/A locus in a nucleic acid sample isolated from a biological sample obtained from said patient, the presence of said at least one methylation associated polymorphism being an indication that said subject has an increased susceptibility to non-response to a hepatitis C treatment, ii) establishing a hepatitis C treatment regimen.

The determination of the methylation associated polymorphism in a subject suffering from chronic or acute hepatitis C will enable the physician to establish the best hepatitis C treatment regimen for said subject (nature, dose and duration of hepatitis C treatment and/or other antiviral drugs). For example, if the above method reveals that at least one methylation associated polymorphism of the invention is present within the 5′ region upstream from the transcription start of the IL28B/A locus in a nucleic acid sample obtained from said subject, indicating that said subject has an increased susceptibility to non-response to a hepatitis C treatment then this subject can be considered as good candidate for newer treatment strategies (such as therapy with higher doses of currently available drugs, longer treatment duration with currently available drugs and/or newer drugs).

Additionally, the Inventors have shown that a subject infected with HCV genotype 1 and/or 4 that carry at least one methylation associated polymorphism within the 5′ region upstream from the transcription start of the IL28B/A locus, will have an increased susceptibility to non-response to a hepatitis C treatment, as shown in both Tables 6, 7 and 9 (These showings demonstrate that the knowledge of both the viral genotype and the host methylation associated polymorphisms are important to predict response to treatment.

Therefore, another aspect of the present invention, and in particular of the method of assessing a susceptibility to non-response to a hepatitis C treatment described supra, comprises the combined determinations of the viral genotype, and of the determination of the polymorphism as described herein, in a subject suffering from chronic hepatitis C, so as to more finely assess the susceptibility to non-response to a hepatitis C treatment or susceptibility to non-spontaneous clearance of HCV infected subjects.

These combined determinations can occur concomitantly or not. If not concomitant, the viral genotype can be assessed first, and then, after a determined time, the determination of the methylation associated polymorphism as described herein occurs. It is also envisioned that the determination of the methylation associated polymorphism as described herein occurs first, and then, after a determined time, the viral genotype is assessed.

Usually, the determined time, which is the time or duration lapsed between the determination of the viral genotype and the determination of the polymorphism (and vice versa) can be comprised between a few seconds and several years.

Also encompassed in the present invention is a kit for determining a susceptibility to non-response to a hepatitis C treatment in a subject suffering from chronic hepatitis C in accordance with the present invention, said kit comprising i) reagents for selectively detecting the presence or absence of at least one methylation associated polymorphism within the 5′ region upstream from the transcription start of the IL28B/A locus, and/or ii) instructions for use.

Further encompassed in the present invention is a kit for determining a susceptibility to non-spontaneous hepatitis C clearance in a subject infected with hepatitis C in accordance with the present invention, said kit comprising i) reagents for selectively detecting the presence or absence of at least one methylation associated polymorphism within the 5′ region upstream from the transcription start of the IL28B/A locus in a nucleic acid sample isolated from a biological sample obtained from the subject and/or ii) instructions for use.

Alternatively, the reagents used in the kits comprise an isolated nucleic acid, preferably a primer, a set of primers, or an array of primers, as described elsewhere herein. The primers may be fixed to a solid substrate. The kits may further comprise a control target nucleic acid and primers. One skilled in the art will, without undue experiments, be able to select the primers in accordance with the usual requirements. The isolated nucleic acids of the kit may also comprise a molecular label or tag.

Usually, the primer, set of primers, or array of primers, are directed to detect the presence or absence of at least one methylation associated polymorphism within the 5′ region upstream from the transcription start of the IL28B/A locus.

The presence or absence of at least one methylation associated polymorphism within the 5′ region upstream from the transcription start of the IL28B/A locus may for example, but not exclusively, be determined using a set of PCR primers or sequencing primers selected from those disclosed in Table 3a.

TABLE 3a

| SNP genotyping | Forward primer | Reverse primer | | |
|---|---|---|---|---|
| NCBI dbSNP ID | (5′→3′) | (3′→5′) | Vic probe | Fam probe |
| rs8099917 | Reference: C_11710096_10²⁾ | | | |
| rs12979860 | TGTACTGAACCAGGGAGCTC | GCGCGGAGTGCAATTCAAC | TGGTTCGCGCCTTC | CTGGTTCACGCCTTC |
| rs4803221 | CCTCAGCGCCTTGGCA | GATGCGGCCGAGTGTCT | CTGCTCTCGCACTACC | CTGCTCTCCCACTACC |
| chr19: 39739154- | CTCCAGCGAGCGGTAGTG | GATGCGGCCGAGTGTCT | TCGCAGAAGGCCC | ATCGCAGCGGCCC |
| 5 TT/-G | | | | |

²⁾Applied Biosystem reference number.

In addition to the primers, set of primers, or array of primers, directed to detect the presence or absence of at least one methylation associated polymorphism within the 5′ region upstream from the transcription start of the IL28B/A locus from a nucleic acid sample isolated from a biological sample obtained from a subject, the reagents of the kit may comprise, for example, an other primer, set of primers, or array of primers, directed to separately detect the viral genotype isolated from a biological sample obtained from a subject. These set of primers, or array of primers used are generally known in the art or may be readily generated knowing the usual requirements.

In additional embodiments, the kits of the present invention comprise various reagents, such as buffers, necessary to practice the methods of the invention, as known in the art.

These reagents or buffers may for example be useful to extract and/or purify the nucleic from the biological sample obtained from the subject.

The kit may also comprise all the necessary material such as microcentrifuge tubes necessary to practice the methods of the invention.

Alternatively, the reagents used in the kits comprise also all the buffers and probes necessary to determine the methylation status of the nucleic acid within the 5′ region upstream from the transcription start of the IL28B/A locus in said nucleic acid sample isolated from a biological sample obtained from the subject.

Numerous methods for analyzing methylation status of a nucleic acid are known in the art and can be used in the methods of the present invention to identify one or more methylation associated polymorphism. In various embodiments, the determining of methylation status in the methods of the invention is performed by one or more techniques selected from the group consisting of a nucleic acid amplification, polymerase chain reaction (PCR), methylation specific PCR, bisulfite pyrosequenceing, single-strand conformation polymorphism (SSCP) analysis, restriction analysis, microarray technology, and proteomics.

As illustrated in the Examples herein, analysis of methylation can be performed by bisulfite genomic sequencing. Bisulfite treatment modifies DNA converting unmethylated, but not methylated, cytosines to uracil. Bisulfite treatment can be carried out using the METHYLEAS Y bisulfite modification kit (Human Genetic Signatures). Exemplary primers for such analysis are set forth in Table 3b.

TABLE 3b

| PCR amplification | | |
|---|---|---|
| Amplicon¹ | Forward | Reverse |
| PAun | 5′-TCAGCCTCGCCTGGTCCTCC-3′ | 5′-TGCTGTGCCTTCACGCTCCG-3′ |
| BSPA1 | 5′-GGGTTAATTTTGTATTGTTTGTTGTATT-3′ | 5′-CATATCCCACCTCCAACTTAAACTA-3′ |
| BSPA2 | 5′-GTTAGTTTAAGTTGGAGGTGGGATAT-3′ | 5′-CAACTTCTCTACATCCTCAAACC-3′ |
| BSPA3 | 5′-GGATTTTAGGATATTTAGAGGATT-3′ | 5′-ATCCTAAAACTACTAAC-3′ |
| BSPA4 | 5′-TTGTATAGTTTGGGATTTTTGGA-3′ | 5′-CAAAATCAATCACAAAAAAAA-3′ |
| BSPA5 | 5′-TTTTTTTGTGATTGATTTTGAGTTT-3′ | 5′-CTATACCCCCTAAAACCCTAACC-3′ |
| BSPA6 | 5′-GTTTTAGGGGGTATAGGGGTTG-3′ | 5′-AAACCCTCTCTTTAACTTCCCTAAC-3′ |
| BSPA7 | 5′-TTAGGGAAGTTAAAGAGAGGGTTTAG-3′ | 5′-ACTCCCACTCTCTCTCCCACTATATA-3′ |

In some embodiments, bisulfite pyrosequencing, which is a sequencing-based analysis of DNA methylation that quantitatively measures multiple, consecutive CpG sites individually with high accuracy and reproducibility, may be used.

Other methods are known in the art for determining methylation status of a DMR, including, but not limited to, array-based methylation analysis, Southern blot analysis, differential methylation hybridization and immunoprecipitation of methylated sequences.

In an array-based methylation analysis, such as methylation profiling arrays, an unmethylated allele of a given DNA sequence is expected to have thymine in place of unmethylated cytosine after treatment with a modifying agent and amplification. Similarly, adenine would be in place of guanine in the complementary strand.

Conversely, these sequences remain unchanged in a methylated allele. Converted amplified DNA can be hybridized to arrayed oligonucleotide probes specifically designed to discriminate between converted and unconverted nucleotides (or their complement) at sites of interest.

In one embodiment, the presence of particular base is determined with the use of a microarray, such as a methylation profiling array. By way of example, sample DNA is bisulfite treated and amplified (for instance by PCR) for a specific region of interest. The amplified product is labeled with Cy5 fluorescent or another dye and hybridized to one or more oligonucleotide probes attached to a substrate. In some examples, an oligonucleotide probe is designed to form a perfect match with a target DNA containing the unmethylated allele. Likewise, in some examples a probe is designed to form a perfect match with the methylated DNA target. Thus, a microarray, such as a methylation profiling array, can be used to determine the methylation status of a particular cytosine, such as a cytosine in the genomic sequence of the 5′ region upstream from the transcription start of the IL28B/A locus.

Methylation profiling arrays may vary in structure, composition, and intended functionality, and may be based on either a macroarray or a microarray format, or a combination thereof. Such arrays can include, for example, at least three, at least 10, at least 25, at least 50, at least 100, or more addresses, usually with a single type of nucleic acid molecule at each address. In the case of macroarrays, sophisticated equipment is usually not required to detect a hybridization signal on the array, though quantification may be assisted by standard scanning and/or quantification techniques and equipment. Thus, macroarray analysis as described herein can be earned out in most hospitals, agricultural and medial research laboratories, universities, or other institutions without the need for investment in specialized and expensive reading equipment.

Examples of substrates for the arrays disclosed herein include glass, Si, Ge, GaAs, GaP, S1O2, S1N4, modified silicon nitrocellulose, polyvinylidene fluoride, polystyrene, polytetrafluoroethylene, polycarbonate, nylon, fiber, or combinations thereof. Array substrates can be stiff and relatively inflexible, such as glass or a supported membrane, or flexible, such as a polymer membrane. One commercially available product line suitable for probe arrays described herein is the Microlite line of MICROTITER® plates available from Dynex Technologies UK (Middlesex, United Kingdom.

In another embodiment, differential methylation hybridization (DMH) may be used to determine the methylation status of the 5′ region upstream from the transcription start of the IL28B/A locus. DHM integrates a high-density, microarray-based screening strategy to detect the presence or absence of methylated CpG dinucleotide genomic fragments. Array-based techniques are used when a number (e.g., >3) of methylation sites in a single region are to be analyzed.

First, CpG dinucleotide nucleic acid fragments from a genomic library are generated, amplified and affixed on a solid support to create a CpG dinucleotide rich screening array. Amplicons are generated by digesting DNA from a sample with restriction endonucleases which digest the DNA into fragments but leaves the methylated CpG islands intact. These amplicons are used to probe the CpG dinucleotide rich fragments affixed on the screening array to identify methylation patterns in the CpG dinucleotide rich regions of the DNA sample. Unlike other methylation analysis methods such as Southern hybridization, bisulfite DNA sequencing and methylation-specific PCR which are restricted to analyzing one gene at a time, DMH utilizes numerous CpG dinucleotide rich genomic fragments specifically designed to allow simultaneous analysis of multiple of methylation-associated genes in the genome (for further details see U.S. Pat. No. 6,605,432).
In yet another embodiment, immunoprecipitation of methylated sequences can be used to isolate sequence-specific methylated nucleic acid fragments. Briefly, nucleic acid such as genomic DNA is sonicated to yield fragments of 200-300 bp. The DNA is then denatured, precleaned with a protein A Fast FlowSepharose) and further incubated with a 5-methylcytidine monoclonal antibody. The complex may be purified using protein A Sepharose and subsequently washed. The immunoprecipitated samples are then analyzed using specific PCR primers.
The invention further contemplates a method of treating a patient for chronic or acute hepatitis C, comprising i) determining the presence or absence of at least one methylation associated polymorphism within the 5′ region upstream from the transcription start of the IL28B/A locus in a nucleic acid sample isolated from a biological sample obtained from said patient, ii) and treating the patient based upon whether said one methylation associated polymorphism within the 5′ region upstream from the transcription start of the IL28B/A locus is associated with increased susceptibility to non-response to hepatitis C treatment.

Generally, the hepatitis C treatment of the invention is either an interferon based treatment or a non interferon based treatment.

In one embodiment, the interferon based treatment is selected from the group comprising IFNα, IFNλ or any pegylated-interferon. Usually, said interferon based treatment is combined with ribavirin. Alternative combinations may include ribavirin and/or antiprotease drugs and/or other antiviral drugs and/or any combination thereof.

In another embodiment, the non-interferon based treatment is selected from the group comprising an antiprotease drug, an antiviral drug and/or any combination thereof.

An example of an antiviral drug of the invention is a cyclophylin inhibitor such as alisporivir.

Examples of an antiprotease drug are selected from the group comprising NS3-4A protease inhibitors such as boceprevir or telaprevir. HCV NS5B inhibitors, such as nucleoside or non-nucleoside NS5B inhibitors, and HCV NS5A inhibitors.

The invention also considers a method of treating a patient for chronic or acute hepatitis C, comprising i) determining the presence or absence of at least one methylation associated polymorphism within the 5′ region upstream from the transcription start of the IL28B/A locus in a nucleic acid sample isolated from a biological sample obtained from said patient, ii) determining the HCV viral genotype in a nucleic acid sample isolated from a biological sample obtained from said patient, iii) and treating the patient based upon whether said one methylation associated polymorphism within the 5′ region upstream from the transcription start of the IL28B/A locus is associated with increased susceptibility to non-response to hepatitis C treatment.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1

### Patients and Methods

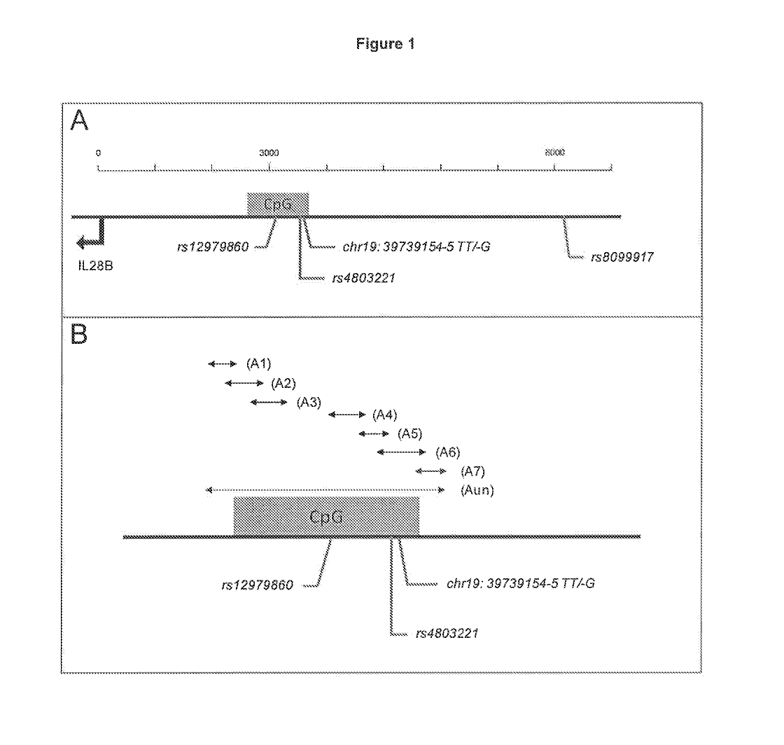

Patients were included from the Swiss Hepatitis C Cohort Study (SCCS), a multicenter study of >3700 HCV-infected patients enrolled at 8 major Swiss hospitals and their local affiliated centers since 2001. Details of patients' selection and data collection were described elsewhere {Prasad et al., 2007; Bochud et al., 2009}. Caucasian patients enrolled in the SCCS before Aug. 1, 2010, with available DNA and written consent for genetic studies were selected. The study included patients with spontaneous HCV clearance (defined as HCV seropositivity and undetectable HCV RNA without previous antiviral treatment) and those who had chronic infection with HCV genotypes 1, 2, 3 or 4 and were assessable for response to therapy with pegylated-interferon alpha and ribavirin, i.e. who received >80% of the recommended dose of each drug. Demographic characteristics including age, sex, HCV risk factors, HCV genotypes, alcohol consumption, markers of chronic infection with the hepatitis B virus and the human immunodeficiency virus (HIV), HCV viral load, liver biopsy data, and HCV treatment were extracted from clinical databases. Sustained viral response was defined as an undetectable HCV RNA in serum more than 24 weeks after treatment termination. Severe fibrosis was considered in patients with a METAVIR score F3. We used the cpgplot tool implemented in EMBOSS (http://www.ebi.ac.uk/Tools/emboss/cpgplot) to determine the presence of CpG island containing sequences up to ˜4000 bp upstream the IL28B start codon. We identified a ˜1000 bp CpG region starting ˜2500 bp upstream the IL28B start codon (FIG. 1). The region was sequenced after bisulfite treatment of DNA in a subset patients chronically infected with HCV genotype 1. The initial screening was performed in 8 patients (4 homozygous for the favorable and 4 homozygous for the unfavorable rs12979860 allele). Polymorphic regions were then sequenced in 26 additional patients (13 homozygous for the favorable and 13 homozygous for the unfavorable rs12979860 allele). Specifically, 250 ng of genomic DNA was treated with sodium bisulfite using EpiTect Bisulfite kit (Qiagen) per manufacturer's recommendations. Seven different fragments covering the CpG region were amplified from bisulfite-modified DNA, using bisulfite sequencing primers (BSP) designed by using MethPrimer (http://www.urogene.org/methprimer/index1.html, Table 3b, FIG. 1). A central zone of about 30 CpG sites was excluded from the analysis since it did not respond to PCR primer design criteria (FIG. 1). Regular non-bisulfite sequencing was performed in 4 patients to obtain reference sequences (2 who were homozygous for the favorable rs12979860 allele and 2 who were homozygous for the unfavorable rs12979860 allele). PCR amplifications from 1 ul of bisulfite-treated genomic DNA consisted on an initial activation of Hot Star Taq DNA polymerase (Qiagen) at 95 for 15 min, followed by 40 cycles at 95 for 30 s, 55 for 30 s, and 72 for 1 min, and 1 cycle at 72 for 10 min. Sequencing was performing using an ABI BigDye Terminator v.3.0 Cycle sequencing kit (Applied Biosystems) and an ABI3130XL Sequencer (Applied Biosystems). BiQ Analyzer software (http://biq-analyzer.bioinf.mpi-sb.mpg.de) and the CpG view program (http://dna.leeds.ac.uk/cpgviewer/) were used to compare the sequences with the reference unmethylated sequence.
SNPs nearby the IL28B locus (rs8099917, rs12979860, chr19: 39739154-5 TT/−G and rs4803221, called IL28B polymorphisms for simplicity) were extracted from a GWA study-generated dataset {Rauch et al., 2010} or genotyped by TaqMan (Applied Biosystems Inc, Foster City, Calif.), using the ABI 7500 Fast real time thermocycler, according to manufacturer's protocols. TaqMan probes and primers were designed and synthesized using Applied Biosystems Inc. software (Table 3). Automated allele calling was performed using SDS software from Applied Biosystems Inc. Patients with at least one missing genotype were excluded from the analyses.
Statistical analyses were performed using Stata (version 11.1, StataCorp LP, College Station, Tex., USA). The association of IL28B polymorphisms with response to treatment and spontaneous clearance were performed by univariate and multivariate logistic regression. Age, duration of infection and body mass index (BMI) were treated as continuous variables. Multiple logistic regression models were adjusted for age, sex, RNA level, fibrosis stage and, whenever appropriate, for viral genotype. For IL28B SNPs, comparisons were made using a dominant model (in which patients carrying one or two copies of minor allele carriers were compared to others), a recessive model (in which patients carrying two copies to the minor allele were compared the others) and an additive model (considering a similar effect for each additional copy of the minor allele). Linkage disequilibrium and Hardy-Weinberg equilibrium test were assessed using the programs pwld and genhw, respectively, both implemented in Stata. Strong LD was defined as a R2>0.7.

TABLE 4

| | Response | Spontaneous |
|---|---|---|
| | to treatment | clearance |
| | N = 631 | N = 112 |
| Proportion | | |
| Male sex | 0.64 | 0.55 |
| White Ethnicity | 1.00 | 1.00 |
| Median age (IQR)¹ | 45 (14) | 38 (9) |
| HCV genotype² | | |
| 1 | 0.45 | NA |
| 2 | 0.12 | NA |
| 3 | 0.33 | NA |
| 4 | 0.09 | NA |
| HCV RNA ≧6 log10 copies/ml³ | 0.50 | NA |
| Median ALT (IU/L, IQR)⁴ | 80 (80) | NA |
| HCV reported risks | | |
| Drug use | 0.38 | NA |
| Invasive procedures/needle stick | 0.22 | NA |
| Transfusion | 0.17 | NA |
| Other/Unknown | 0.22 | NA |
| Alcohol consumption⁵ | | |
| >40 g/d for ≧5 years | 0.20 | NA |
| Diabetes | 0.08 | NA |
| Median BMI (kg/m², IQR)⁶ | 23.4 (5.1) | NA |
| HIV+⁷ | 0.05 | NA |
| Chronic HBV infection⁸ | 0.02 | NA |
| Biopsy | | |
| Histological activity⁹ | | |
| 0 (none or minimal) | 0.34 | NA |
| 1 (mild, moderate or severe) | 0.66 | NA |
| Fibrosis stage (Metavir)¹⁰ | | |
| 0-1 | 0.28 | NA |
| 2-4 | 0.72 | NA |
| Steatosis¹¹ | 0.67 | NA |

Numbers are the proportion of patients with indicated characteristics.
¹Age at treatment start for patients with chronic infection, at cohort entry for those with spontaneous clearance
²HCV genotypes are missing in most patients with spontaneous clearance
³Median RNA before treatment was 6 log10 copies/ml, data was missing in 82 patients.
⁴ALT values before treatment was missing in 196 patients.
⁵Alcohol consumption data before treatment was missing in 9 patients.
⁶BMI before/during treatment was missing in 95 patients.
⁷HIV serostatus was missing in 110 patients.
⁸HBV serostatus was missing in 131 patients
⁹Histological activity before treatment was missing in 183 patients.
¹⁰Fibrosis stage before treatment was missing in 181 patients.
¹¹Steatosis data before treatment were missing in 125 patients.

### Example 2

### Results

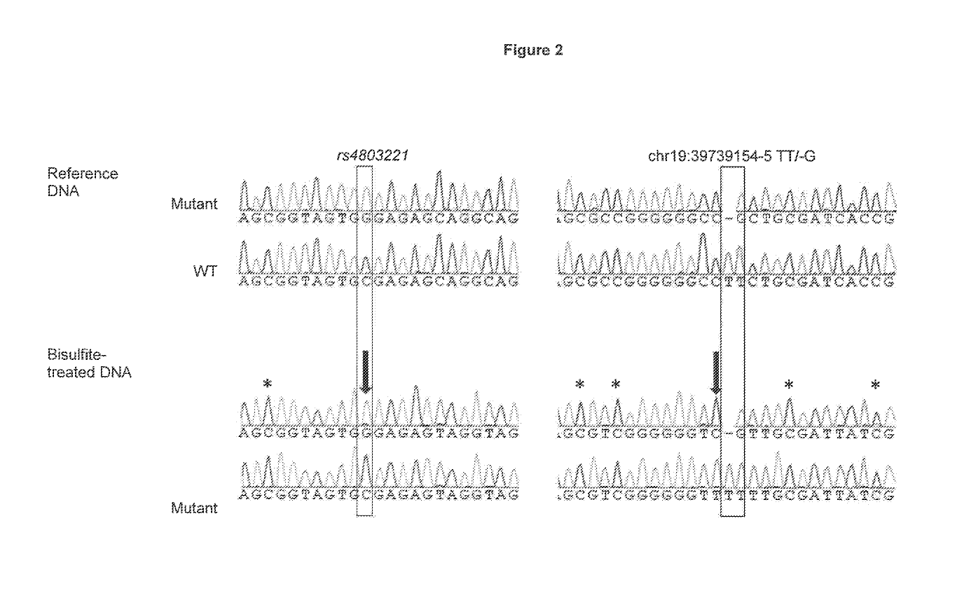

The profile of methylation in a CpG region located (˜2500 to ˜3500 bp upstream IL28B start codon, FIG. 1) was analyzed by bisulfite-sequencing in SCCS patients. Overall, the methylation profile in the CpG region was conserved. Only two loci revealed different methylation patterns (FIG. 2). In both cases, the methylation depended on polymorphisms in the native DNA sequence. The first locus was located ˜3500 bp upstream the IL28B start codon, where a C to G substitution, previously described as rs4803221, resulted in a loss of methylation. The second locus was located ˜3550 bp upstream IL28B start codon, where a T deletion (previously described as rs67272382 and rs67272383 followed by a T to G substitution (previously described as rs74597329) resulted in a gain of methylation. In all 34 sequenced patients, the deletion and substitution were always present or absent together, suggesting that rs67272382 and rs74597329 can be considered as a single polymorphism, called chr19: 39739154-5 TT/−G deletion/substitution.
The presence of the methylation pattern-associated polymorphisms (rs4803221 and chr19: 39739154-5 TT/−G), as well as previously known SNPs (rs8099917 and rs12979860), was explored in 631 patients with chronic HCV infection and 119 with spontaneous clearance. The demographic and clinical characteristics of these patients are shown in Table 4. The minor allele frequencies (MAFs) of rs12979860, rs4803221, chr19: 39739154-5 TT/−G and rs8099917 were 0.36, 0.22, 0.35 and 0.23 respectively (Table 5); rs8099917 and rs4803221 were in strong LD (R2=0.70), as well as were rs12979860 and chr19: 39739154-5 TT/−G (R2=0.75).
First, we assessed the association of IL28B polymorphisms with spontaneous viral clearance. All polymorphisms were significantly associated with reduced clearance (Table 6). The strongest and most significant association was found for chr19: 39739154-5 TT/−G (OR=0.38, 95% CI 0.27-0.55, P=2.23E-7), followed by rs12979860 (OR=0.42, 95% CI 0.30-0.60, P=1.84E-06), rs4803221 (OR=0.40, 95% CI 0.26-0.62, P=4.54E-05) and rs8099917 (OR=0.43, 95% CI 0.28-0.66, P=1.12E-04). When both rs12979860 and chr19: 39739154-5 TT/−G polymorphisms were included in the same model, the association of rs12979860 with clearance disappeared (OR=0.76, 95% CI 0.44-1.32, P=0.3), while the association of chr19: 39739154-5 TT/−G remained significant (OR=0.47, 95% CI 0.27-0.84, P=0.01) (Table 11). These data confirmed that chr19: 39739154-5 TT/−G is a better marker than rs12979860.
Second, we assessed the association of IL28B polymorphisms with response to pegylated-interferon alpha and ribavirin therapy among chronically infected patients. When considering all viral genotypes together, all polymorphisms were associated with response to treatment (Table 7). The strongest and most significant association was found for chr19: 39739154-5 TT/−G (OR=0.42, 95% CI 0.33-0.55, P=1.49E-10), followed by rs12979860 (P=4.02E-08), rs8099917 (P=5.02E-08) and rs4803221 (P=2.11E-07). Chr19: 39739154-5 TT/−G still provided the strongest and most significant association in a multivariate model, after adjustment for age, sex, RNA level, fibrosis stage and viral genotype (P=7.40E-07), followed by rs8099917 (P=1.08E-06), rs4803221 (P=1.3E-06) and rs12979860 (P=1.45E-05). When different combinations of polymorphisms were included together into the models, the association with chr19: 39739154-5 TT/−G remained significant, while the association with rs12979860 and rs4803221 disappeared, and the association with rs8099917 was borderline (Table 11).
When considering patients infected with HCV genotype 1 and 4, the strongest and most significant association was also found for chr19: 39739154-5 TT/−G (OR=0.27, 95% CI 0.18-0.40, P=1.52E-10, Table 8), followed by rs12979860 (P=1.49E-08), rs8099917 (P=4.94E-08) and rs4803221 (P=5.03E-07). Chr19: 39739154-5 TT/−G still provided the strongest and most significant association in a multivariate model, after adjustment for age, sex, RNA level, fibrosis stage and viral genotype (P=8.88E-08), followed by rs8099917 (P=4.47E-07), rs12979860 (P=6.65E-07) and rs4803221 (P=1.11E-06). In particular, chr19: 39739154-5 TT/−G had a better ability than rs12979860 to discriminate SVR rates, especially in patients homozygous for the rare allele. The SVR proportion was 0.70, 0.36 and 0.16 for patients carrying the chr19: 39739154-5 TT/TT, TT/−G and −G/−G genotypes, compared to 0.69, 0.36 and 0.24 for those carrying rs12979860 C/C, C/T and T/T genotypes. Similar results were observed when the analyses were restricted to patients infected with HCV viral genotype 1 only (Table 9).
When considering patients infected with HCV genotype 2 and 3, IL28B polymorphisms did not show significant associations with response to treatment (Table 10). However, there was a borderline association for chr19: 39739154-5 TT/−G when the analyses were performed using a recessive model (OR=0.46, 95% CI 0.21-1.05, P=6.39E-02). This association was not confirmed in the multivariate model (P=5.38E-01).

TABLE 5
Linkage disequilibrium between IL28B-associated polymorphisms
among 631 patients with chronic hepatitis C

| | | | |
|---|---|---|---|
| | | chr19: | |
| | | 39739154-5 | |
| rs12979860 | rs4803221 | TT/-G | rs8099917 |

A. Lewontin's D′

| | | | | |
|---|---|---|---|---|
| rs12979860 | 0.36 | | | |
| rs4803221 | 0.86 | 0.22 | | |
| chr19: 39739154-5 | 0.89 | 0.92 | 035 | |
| TT/-G | | | | |
| rs8099917 | 0.93 | 0.84 | 0.77 | 0.23 |

B. R-squared

| | | | | |
|---|---|---|---|---|
| rs12979860 | 0.36 | | | |
| rs4803221 | 0.38 | 0.22 | | |
| chr19: 39739154-5 | 0.75 | 0.45 | 0.35 | |
| TT/-G | | | | |
| rs8099917 | 0.45 | 0.70 | 0.33 | 0.23 |

Off-diagonal elements are estimates of Lewontin's D′ (A) and R-squared (B), both assuming Hardy-Weinberg equilibrium. Diagonal elements are shown in bold and indicate relative frequencies of the minor allele. Calculated using pwld (Stata 11.1 for Windows, College Station, TX, USA).

TABLE 6
Association of IL28B polymorphisms with spontaneous clearance of HCV

| | | Chronic Infection | | Spontaneous Clearance | | | |
|---|---|---|---|---|---|---|---|
| | Geno- | (N = 631) | | (N = 112) | | | |
| SNP | type | N | Prop. | N | Prop. | OR (95% CI) | P |
| rs12979860 | C/C | 221 | 0.35 | 68 | 0.61 | | |
| | C/T | 333 | 0.53 | 38 | 0.34 | | |
| | T/T | 77 | 0.12 | 6 | 0.05 | 0.42 (0.30-0.60) | 1.84E−06 |
| rs4803221 | C/C | 359 | 0.57 | 88 | 0.79 | | |
| | C/G | 239 | 0.38 | 22 | 0.20 | | |
| | G/G | 33 | 0.05 | 2 | 0.02 | 0.40 (0.26-0.62) | 4.54E−05 |
| chr19: 39739154-5 | TT/TT | 234 | 0.37 | 72 | 0.64 | | |
| TT/−G | TT/−G | 321 | 0.51 | 36 | 0.32 | | |
| | −G/−G | 76 | 0.12 | 4 | 0.04 | 0.38 (0.27-0.55) | 2.23E−07 |
| rs8099917 | T/T | 355 | 0.56 | 88 | 0.79 | | |
| | T/G | 244 | 0.39 | 20 | 0.18 | | |
| | G/G | 32 | 0.05 | 4 | 0.04 | 0.43 (0.28-0.66) | 1.12E−04 |

SNP stands for single nucleotide polymorphism, OR for odds ratio and CI for confidence interval. Odd ratios and P values are for an additive model, accounting for increased effect of 1 or 2 copies of the minor allele.

TABLE 7
Association of IL28B polymorphisms with response to pegylated interferon alpha and
ribavirin therapy in patients chronically infected with HCV genotype 1, 2, 3 and 4

| | | | | Additive² univariate | | Additive² multivariate | |
|---|---|---|---|---|---|---|---|
| | Geno- | | Prop. | models (N = 631)³ | | models (N = 442)³ | |
| SNP | type | N | SVR¹ | OR (95% CI) | P | OR (95% CI) | P |
| rs12979860 | C/C | 221 | 0.76 | | | | |
| | C/T | 333 | 0.56 | | | | |
| | T/T | 77 | 0.44 | 0.48 (0.37-0.63) | 4.02E−08 | 0.41 (0.28-0.62) | 1.45E−05 |
| rs4803221 | C/C | 359 | 0.70 | | | | |
| | C/G | 239 | 0.51 | | | | |
| | G/G | 33 | 0.39 | 0.48 (0.37-0.64) | 2.11E−07 | 0.33 (0.21-0.52) | 1.30E−06 |
| chr19: 39739154-5 | TT/TT | 234 | 0.77 | | | | |
| TT/−G | TT/−G | 321 | 0.55 | | | | |
| | −G/−G | 76 | 0.39 | 0.42 (0.33-0.55) | 1.49E−10 | 0.35 (0.23-0.53) | 7.40E−07 |
| rs8099917 | T/T | 355 | 0.72 | | | | |
| | T/G | 244 | 0.49 | | | | |
| | G/G | 32 | 0.47 | 0.46 (0.35-0.61) | 5.02E−08 | 0.33 (0.21-0.52) | 1.08E−06 |

SNP stands for single nucleotide polymorphism, OR for odds ratio and CI for confidence interval.
¹Data indicate the proportion of patients with sustained viral response (SVR) for indicated host and viral genotypes.
²Odd ratios and P values are for an additive model, accounting for increased effect of 1 or 2 copies of the minor allele.
³Multivariate models are adjusted for age, sex, RNA level, fibrosis stage and viral genotype. Multivariate models included a smaller number of patients, due to missing covariates in some patients.

TABLE 8
Association of IL28B polymorphisms with response to pegylated interferon alpha and
ribavirin therapy in patients chronically infected with HCV genotype 1 and 4

| | | | | Additive² univariate | | Additive² multivariate | |
|---|---|---|---|---|---|---|---|
| | Geno- | | Prop. | models (N = 342) | | models (N = 236)³ | |
| SNP | type | N | SVR¹ | OR (95% CI) | P | OR (95% CI) | P |
| rs12979860 | C/C | 101 | 0.69 | | | | |
| | C/T | 195 | 0.36 | | | | |
| | T/T | 46 | 0.24 | 0.33 (0.22-0.48) | 1.49E−08 | 0.26 (0.15-0.44) | 6.65E−07 |
| rs4803221 | C/C | 175 | 0.57 | | | | |
| | C/G | 149 | 0.35 | | | | |
| | G/G | 18 | 0.06 | 0.36 (0.24-0.53) | 5.03E−07 | 0.23 (0.13-0.42) | 1.11E−06 |
| chr19: 39739154-5 | TT/TT | 107 | 0.70 | | | | |
| TT/−G | TT/−G | 192 | 0.36 | | | | |
| | −G/−G | 43 | 0.16 | 0.27 (0.18-0.40) | 1.52E−10 | 0.21 (0.12-0.37) | 8.88E−08 |
| rs8099917 | T/T | 176 | 0.60 | | | | |
| | T/G | 148 | 0.29 | | | | |
| | G/G | 18 | 0.22 | 0.32 (0.21-0.48) | 4.94E−08 | 0.21 (0.12-0.39) | 4.47E−07 |

SNP stands for single nucleotide polymorphism, OR for odds ratio and CI for confidence interval.
¹Data indicate the proportion of patients with sustained viral response (SVR) for indicated host and viral genotypes.
²Odd ratios and P values are for an additive model, accounting for increased effect of 1 or 2 copies of the minor allele.
³Multivariate models are adjusted for age, sex, RNA level, fibrosis stage and viral genotype. Multivariate models include a smaller number of patients, due to missing covariates in some patients.

TABLE 9
Association of IL28B polymorphisms with response to pegylated interferon alpha and
ribavirin therapy in patients chronically infected with HCV genotype 1 alone

| | | | | Additive² univariate | | Additive² multivariate | |
|---|---|---|---|---|---|---|---|
| | Geno- | | Prop. | models (N = 271) | | models (N = 201)³ | |
| SNP | type | N | SVR¹ | OR (95% CI) | P | OR (95% CI) | P |
| rs12979860 | C/C | 85 | 0.66 | | | | |
| | C/T | 166 | 0.36 | | | | |
| | T/T | 34 | 0.26 | 0.37 (0.24-0.57) | 3.90E−06 | 0.28 (0.15-0.51) | 2.67E−05 |
| rs4803221 | C/C | 150 | 0.65 | | | | |
| | C/G | 122 | 0.34 | | | | |
| | G/G | 13 | 0.01 | 0.40 (0.26-0.62) | 4.44E−05 | 0.24 (0.13-0.47) | 2.04E−05 |
| chr19: 39739154-5 | TT/TT | 89 | 0.67 | | | | |
| TT/−G | TT/−G | 164 | 0.36 | | | | |
| | −G/−G | 32 | 0.16 | 0.29 (0.18-0.44) | 3.02E−08 | 0.21 (0.11-0.40) | 2.62E−06 |
| rs8099917 | T/T | 150 | 0.57 | | | | |
| | T/G | 121 | 0.29 | | | | |
| | G/G | 14 | 0.29 | 0.38 (0.24-0.59) | 1.60E−05 | 0.22 (0.12-0.43) | 7.19E−06 |

SNP stands for single nucleotide polymorphism, OR for odds ratio and CI for confidence interval.
¹Data indicate the proportion of patients with sustained viral response (SVR) for indicated host and viral genotypes.
²Odd ratios and P values are for an additive model, accounting for increased effect of 1 or 2 copies of the minor allele.
³Multivariate models are adjusted for age, sex, RNA level, fibrosis stage and, whenever appropriate, viral genotype. Multivariate models include a smaller number of patients (N = 201), due to missing covariates some patients.

TABLE 10
Association of IL28B polymorphisms with response to pegylated interferon alpha and
ribavirin therapy in patients chronically infected with HCV genotype 2 and 3

| | | | | Recessive² univariate | | Recessive² multivariate | |
|---|---|---|---|---|---|---|---|
| | Geno- | | Prop. | models (N = 289) | | models (N = 186)³ | |
| SNP | type | N | SVR¹ | OR (95% CI) | P | OR (95% CI) | P |
| rs12979860 | C/C | 120 | 0.81 | | | | |
| | C/T | 138 | 0.84 | | | | |
| | T/T | 31 | 0.74 | 0.61 (0.26-1.44) | 2.59E−01 | 0.90 (0.45-1.80) | 7.63E−01 |
| rs4803221 | C/C | 184 | 0.83 | | | | |
| | C/G | 90 | 0.79 | | | | |
| | G/G | 15 | 0.80 | 0.89 (0.24-3.28) | 8.65E−01 | 0.73 (0.32-1.66) | 4.50E−01 |
| chr19: 39739154-5 | TT/TT | 127 | 0.83 | | | | |
| TT/−G | TT/−G | 129 | 0.84 | | | | |
| | −G/−G | 33 | 0.70 | 0.46 (0.21-1.05) | 6.39E−02 | 0.80 (0.40-1.61) | 5.38E−01 |
| rs8099917 | T/T | 179 | 0.83 | | | | |
| | T/G | 96 | 0.79 | | | | |
| | G/G | 14 | 0.79 | 0.81 (0.22-3.03) | 7.60E−01 | 0.72 (0.34-1.55) | 4.04E−01 |

SNP stands for single nucleotide polymorphism, OR for odds ratio and CI for confidence interval.
¹Data indicate the proportion of patients with sustained viral response (SVR) for indicated host and viral genotypes.
²Odd ratios and P values are for a recessive model, comparing the presence of 2 copies versus 0 or 1 copies of the minor allele.
³Multivariate models are adjusted for age, sex, RNA level, fibrosis stage and, whenever appropriate, viral genotype. Multivariate models include a smaller number of patients due to missing covariates in some patients.

TABLE 11
Models combining different IL28B polymorphisms

| | Polymorphisms analyzed | | Polymorphisms included | |
|---|---|---|---|---|
| | in separate models | | in the same model | |
| SNP | OR (95% CI) | P | OR (95% CI) | P |

Spontaneous clearance

| | | | | |
|---|---|---|---|---|
| rs12979860 | 0.42 (0.30-0.60) | 1.84E−06 | 0.88 (0.46-1.69) | 7.03E−01 |
| rs4803221 | 0.40 (0.26-0.62) | 4.54E−05 | 0.88 (0.41-1.89) | 7.39E−01 |
| chr19: 39739154-5 TT/-G | 0.38 (0.27-0.55) | 2.23E−07 | 0.50 (0.26-0.98) | 4.30E−02 |
| rs8099917 | 0.43 (0.28-0.66) | 1.12E−04 | 0.80 (0.39-1.65) | 5.44E−01 |
| SNPs by pairs | | | | |
| chr19: 39739154-5 TT/-G | | | 0.47 (0.27-0.84) | 1.10E−02 |
| rs12979860 | | | 0.76 (0.44-1.32) | 3.35E−01 |
| chr19: 39739154-5 TT/-G | | | 0.46 (0.29-0.73) | 1.02E−03 |
| rs4803221 | | | 0.72 (0.41-1.27) | 2.60E−01 |
| chr19: 39739154-5 TT/-G | | | 0.45 (0.29-0.68) | 1.67E−04 |
| rs8099917 | | | 0.71 (0.44-1.14) | 1.54E−01 |

Response to treatment (all viral
genotypes)¹

| | | | | |
|---|---|---|---|---|
| rs12979860 | 0.41 (0.28-0.62) | 1.45E−05 | 1.47 (0.83-2.61) | 1.85E−01 |
| rs4803221 | 0.33 (0.21-0.52) | 1.30E−06 | 1.37 (0.76-2.47) | 2.98E−01 |
| chr19: 39739154-5 TT/-G | 0.35 (0.23-0.53) | 7.40E−07 | 0.36 (0.20-0.63) | 4.20E−04 |
| rs8099917 | 0.33 (0.21-0.52) | 1.08E−06 | 0.49 (0.27-0.88) | 1.73E−02 |
| SNPs by pairs | | | | |
| chr19: 39739154-5 TT/-G | | | 0.41 (0.25-0.67) | 3.16E−04 |
| rs12979860 | | | 1.03 (0.63-1.97) | 9.13E−01 |
| chr19: 39739154-5 TT/-G | | | 0.47 (0.32-0.68) | 7.69E−05 |
| rs4803221 | | | 0.81 (0.55-1.21) | 3.20E−01 |
| chr19: 39739154-5 TT/-G | | | 0.51 (0.36-0.71) | 7.95E−05 |
| rs8099917 | | | 0.67 (0.47-0.96) | 3.02E−02 |

Response to treatment (viral
genotypes 1 & 4)¹

| | | | | |
|---|---|---|---|---|
| rs12979860 | 0.26 (0.15-0.44) | 6.65E−07 | 1.04 (0.53-2.05) | 9.09E−01 |
| rs4803221 | 0.23 (0.13-0.42) | 1.11E−06 | 1.25 (0.59-2.64) | 5.62E−01 |
| chr19: 39739154-5 TT/-G | 0.21 (0.12-0.37) | 8.88E−08 | 0.33 (0.16-0.66) | 1.82E−03 |
| rs8099917 | 0.21 (0.12-0.39) | 4.47E−07 | 0.44 (0.21-0.90) | 2.61E−02 |
| SNPs by pairs | | | | |
| chr19: 39739154-5 TT/-G | | | 0.35 (0.19-0.64) | 7.07E−04 |
| rs12979860 | | | 0.72 (0.40-1.30) | 2.76E−01 |
| chr19: 39739154-5 TT/-G | | | 0.34 (0.21-0.56) | 1.91E−05 |
| rs4803221 | | | 0.67 (0.40-1.12) | 1.27E−01 |
| chr19: 39739154-5 TT/-G | | | 0.36 (0.23-0.56) | 8.37E−06 |
| rs8099917 | | | 0.51 (0.32-0.81) | 4.68E−03 |

SNP stands for single nucleotide polymorphism, OR for odds ratio and CI for confidence interval.
¹adjusted for age, sex, RNA level, fibrosis stage and viral genotype.

### REFERENCES

Rauch A, Kutalik Z, Descombes P, Cai T, Di Iulio J, Mueller T, et al. Genetic variation in IL28B is associated with chronic hepatitis C and treatment failure: a genome-wide association study. Gastroenterology 2010,138:1338-1345, 1345 e1331-1337.
Ge D, Fellay J, Thompson A J, Simon J S, Shianna K V, Urban T J, et al. Genetic variation in IL28B predicts hepatitis C treatment-induced viral clearance. Nature 2009,461:399-401.
Suppiah V, Moldovan M, Ahlenstiel G, Berg T, Weltman M, Abate M L, et al. IL28B is associated with response to chronic hepatitis C interferon-alpha and ribavirin therapy. Nat Genet 2009, 41:1100-1104.
Tanaka Y, Nishida N, Sugiyama M, Kurosaki M, Matsuura K, Sakamoto N, et al. Genome-wide association of IL28B with response to pegylated interferon-alpha and ribavirin therapy for chronic hepatitis C. Nat Genet 2009, 41:1105-1109.
Ank N, West H, Bartholdy C, Eriksson K, Thomsen A R, Paludan S R. Lambda interferon (IFN-lambda), a type III IFN, is induced by viruses and IFNs and displays potent antiviral activity against select virus infections in vivo. J Virol 2006,80:4501-4509.
Robek M D, Boyd B S, Chisari F V. Lambda interferon inhibits hepatitis B and C virus replication. J Virol 2005, 79:3851-3854.
Lange C M, Zeuzem S. IL28B single nucleotide polymorphisms in the treatment of hepatitis C. Journal of hepatology 2011.
Dill M T, Duong F H, Vogt J E, Bibert S, Bochud P Y, Terracciano L, et al. Interferon-induced gene expression is a stronger predictor of treatment response than IL28B genotype in patients with hepatitis C. Gastroenterology 2011, 140:1021-1031.
Honda M, Sakai A, Yamashita T, Nakamoto Y, Mizukoshi E, Sakai Y, et al. Hepatic ISG expression is associated with genetic variation in interleukin 28B and the outcome of IFN therapy for chronic hepatitis C. Gastroenterology 2010,139:499-509.
Urban T J, Thompson A J, Bradrick S S, Fellay J, Schuppan D, Cronin K D, et al. IL28B genotype is associated with differential expression of intrahepatic interferon-stimulated genes in patients with chronic hepatitis C. Hepatology 2010,52:1888-1896.
di Iulio J, Ciuffi A, Fitzmaurice K, Kelleher D, Rotger M, Fellay J, et al. Estimating the net contribution of interleukin-28B variation to spontaneous hepatitis C virus clearance. Hepatology 2011, 53:1446-1454.
Prasad L, Spicher V M, Zwahlen M, Rickenbach M, Helbling B, Negro F. Cohort Profile: the Swiss Hepatitis C Cohort Study (SCCS). Int J Epidemiol 2007,36:731-737.
Bochud P Y, Cai T, Overbeck K, Bochud M, Dufour J F, Mullhaupt B, et al. Genotype 3 is associated with accelerated fibrosis progression in chronic hepatitis C. J Hepatol 2009,51:655-666.

1. A method of determining a susceptibility of non-response to a hepatitis C treatment in a subject suffering from chronic hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

2. The method of claim 1, wherein said at least one methylation associated polymorphism is in a CpG region.

3. The method of claim 2, wherein the CpG region is located on chromosome 19 between base pairs 39738000 and 39739500.

4. The method of claim 1, wherein the presence of the at least one methylation associated polymorphism is an indication that said subject has an increased or decreased susceptibility to non-response to a hepatitis C treatment.

5. The method of claim 4, wherein the at least one methylation associated polymorphism, which presence is an indication that said subject has an increased susceptibility to nonresponse to a hepatitis C treatment, is selected from the group consisting of rs67272382 T/−, rs67272382−/−, rs74597329 T/G, rs74597329 G/G, rs4803221 C/G and rs4803221 G/G.

6. The method of claim 1, wherein the at least one methylation associated polymorphism is in complete or strong linkage disequilibrium with at least one SNP selected from the group consisting of rs12979860 and rs8099917.

7. The method of claim 1, wherein the hepatitis C treatment is an interferon based treatment or a non-interferon based treatment.

8. The method of claim 7, wherein the interferon based treatment is selected from the group consisting of IFNα, IFNλ and any pegylated-interferon.

9. The method of claim 7, wherein the non-interferon based treatment is selected from the group consisting of an antiprotease drug, an antiviral drug and any combination thereof.

10. The method of claim 7, wherein the interferon based treatment is combined with ribavirin and/or an antiprotease drug and/or an antiviral drug and/or any combination thereof.

11. The method of claim 7, wherein the interferon based treatment is combined with an antiprotease drug and/or an antiviral drug and/or any combination thereof.

12. The method of claim 9, wherein the antiviral drug is a cyclophylin inhibitor.

13. The method of claim 9, wherein the antiprotease drug is selected from the group consisting of an NS3-4A protease inhibitor, an HCV NS5B inhibitor and an HCV NS5A inhibitor.

14. The method of claim 1, wherein said chronic hepatitis C is caused by a viral genotype 1, 2, 3, 4, 5 or 6 of HCV.

15. The method of claim 1, further comprising determining the HCV viral genotype in a nucleic acid sample isolated from a biological sample of said subject.

16. A method of determining a susceptibility to non-spontaneous hepatitis C clearance in a subject infected with hepatitis C, said method comprising determining, in a nucleic acid sample isolated from a biological sample obtained from said subject, the presence or absence of at least one methylation associated polymorphism within the 5′ region upstream from the transcription start of the IL28B/A locus.

17. The method of claim 16, wherein said at least one methylation associated polymorphism is in a CpG region.

18. The method of claim 17, wherein the CpG region is located on chromosome 19 between base pairs 39738000 and 39739500.

19. The method of claim 16, wherein the presence of the at least one methylation associated polymorphism is an indication that said subject has an increased susceptibility to non-spontaneous hepatitis C clearance.

20. The method of claim 16, wherein the at least one methylation associated polymorphism, which presence is an indication that said subject has an increased susceptibility to non-spontaneous hepatitis C clearance, is selected from the group consisting of rs67272382 T/−, rs67272382−/−, rs74597329 T/G, rs74597329 G/G, rs4803221 C/G and rs4803221 G/G.

21. The method of claim 16, wherein the at least one methylation associated polymorphism is in complete or strong linkage disequilibrium with at least one SNP selected from the group consisting of rs12979860, rs12979860, rs12979860, rs8099917, rs8099917, and rs8099917.

22. The method of claim 16, wherein said chronic hepatitis C is caused by a viral genotype 1, 2, 3, 4, 5 or 6 of HCV.

23. The method of claim 16, further comprising determining the HCV viral genotype in a nucleic acid sample isolated from a biological sample of said subject.

24. A kit for determining a susceptibility to non-response to a hepatitis C treatment in a subject suffering from chronic hepatitis C according to the method of claim 1, said kit comprising i) reagents for selectively detecting the presence or absence of at least one methylation associated polymorphism within the 5′ region upstream from the transcription start of the IL28B/A locus ii) instructions for use.

25. A kit for determining a susceptibility to non-spontaneous hepatitis C clearance in a subject infected with hepatitis C according to the method of claim 1, said kit comprising i) reagents for selectively detecting the presence or absence of at least one methylation associated polymorphism within the 5′ region upstream from the transcription start of the IL28B/A locus and ii) instructions for use.

26. The kit of claim 24, wherein the reagents further comprise another primer, set of primers, or array of primers, directed to detect the viral genotype.

27. A method of treating a patient for hepatitis C, comprising
i) determining the presence or absence of at least one methylation associated polymorphism within the 5′ region upstream from the transcription start of the IL28B/A locus in a nucleic acid sample isolated from a biological sample obtained from said patient,
ii) and treating the patient based upon whether said one methylation associated polymorphism within the 5′ region upstream from the transcription start of the IL28B/A locus is associated with increased susceptibility to non-response to hepatitis C treatment.

28. A method of treating a patient for hepatitis C, comprising
i) determining the presence or absence of at least one methylation associated polymorphism within the 5′ region upstream from the transcription start of the IL28B/A locus in a nucleic acid sample isolated from a biological sample obtained from said patient,
ii) determining the HCV viral genotype in a nucleic acid sample isolated from a biological sample obtained from said patient,
iii) and treating the patient based upon whether said one methylation associated polymorphism within the 5′ region upstream from the transcription start of the IL28B/A locus is associated with increased susceptibility to non-response to hepatitis C treatment.

29. A method of assessing a susceptibility to non-response to a hepatitis C treatment in a subject suffering from hepatitis C, said method comprising:
i) distinguishing in said subjects those having a susceptibility to non-response to a hepatitis C treatment by determining the presence or absence of at least one methylation associated polymorphism within the 5′ region upstream from the transcription start of the IL28B/A locus in a nucleic acid sample isolated from a biological sample obtained from said patient, the presence of said at least one methylation associated polymorphism being an indication that said subject has an increased susceptibility to non-response to a hepatitis C treatment, ii) establishing a hepatitis C treatment regimen.

30. A method of assessing a susceptibility to non-response to a hepatitis C treatment in a subject suffering from hepatitis C, said method comprising:
i) distinguishing in said subjects those having a susceptibility to non-response to a hepatitis C treatment by determining
the presence or absence of at least one methylation associated polymorphism within the 5′ region upstream from the transcription start of the IL28B/A locus in a nucleic acid sample isolated from a biological sample obtained from said subject, the presence of said one methylation associated polymorphism being an indication that said subject has an increased susceptibility to non-response to a hepatitis C treatment, and
the HCV viral genotype, the presence of genotype 1 and/or 4 being an indication that said subject has an increased susceptibility to non-response to a hepatitis C treatment,
ii) establishing a hepatitis C treatment regimen.

31. The method of claim 27, wherein the subject suffers from chronic hepatitis C or acute hepatitis C.
